(19) United States
(12) Patent Application Publication
(10) Pub. No.: US 2011/0196458 A1
(43) Pub. Date: Aug. 11, 2011

(54) THERAPY GARMENT

(76) Inventor: LARRY T. BRATCHER, JR., HILLSBOROUGH, NC (US)

(21) Appl. No.: 12/701,181

(22) Filed: Feb. 5, 2010

(51) Int. Cl. A61F 7/00

(52) U.S. Cl. 607/108; 607/112

(57) ABSTRACT

A therapy garment is provided for providing temperature or pressure therapy to a user. The therapy garment may comprise a main body member, a pair of leg sections, and at least one pocket and may be adapted to be worn by the user so that the pockets align with the region of the user's body that is to receive therapy.

### BACKGROUND

This invention relates generally to a therapy garment, and more particularly to a lower body garment including pockets at predetermined locations for accommodating therapeutic temperature elements.

Major muscle groups of the lower body can be painful or fatigued after exercise, due in part to lactic acid buildup. These same muscle groups can also be slow to heal after an injury or after surgery. Temperature therapy, whether it be heating or cooling, may reduce pain and fatigue in muscles and may enhance healing of injuries. Applying pressure to muscle groups may also reduce any potential swelling due to exercise or injury and provide a therapeutic effect. Unfortunately, it is difficult to apply either temperature or pressure therapy to multiple muscle groups at one time and still allow the user to remain mobile.

Previously, some individuals attempted to reduce pain and fatigue in lower body muscles by standing or sitting in an ice bath. This method presents numerous problems including discomfort, lack of mobility, and difficulty in setting up the ice bath. An ice bath does not target the specific areas in need of therapy. Further, this method of treatment does not provide pressure therapy.

For the foregoing reasons, there is a need for a therapy garment that can provide targeted temperature or pressure therapy to multiple muscle groups of the lower body at the same time while still allowing the user to be mobile.

### SUMMARY

A therapy garment is provided for applying localized temperature therapy to a body of a user. The therapy garment comprises a tubular main body member, a pair of leg sections, and at least one pocket. The main body member encircles the torso of the user and has a pair of openings to accommodate the limbs of the user. The pair of leg sections are attached to a portion of the pair of openings of the main body member and adapted to encircle the legs of the user. The at least one pocket is located on an outer face of the therapy garment and adapted to overlay with a region on the user that is to receive temperature therapy. In an alternative embodiment, the therapy garment may further comprise a removable temperature therapy element that fits within the at least one pocket. The removable temperature therapy element may selectively provide heating or cooling to the region of the body of the user that is to receive temperature therapy.

In alternative embodiments, the therapy garment further comprises at least one compression region adapted to apply therapeutic pressure to the body of the user. The compression region may apply a consistent level of pressure to the region. Preferably, the compression region may be comprised of compression fabric.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature and not as restrictive. The full scope of the subject matter is set forth in the claims.

### BRIEF DESCRIPTION OF THE DRAWINGS

For a more complete understanding of the present invention, reference should now be had to the embodiments shown in the accompanying drawings and described below. In the drawings:

FIG. 1 is a front view of a user wearing an embodiment of a therapy garment.

FIG. 2 is a right side view of the user wearing the therapy garment as shown in FIG. 1, the left-side view being a mirror image.

FIG. 3 is a front elevation view of the therapy garment as shown in FIG. 1.

FIG. 4 is a rear elevation view of the therapy garment as shown in FIG. 1.

FIG. 5 is an exploded right side elevation view of the therapy garment as shown in FIGS. 3 and 4 and an embodiment of a removable temperature therapy element.

### DESCRIPTION

Certain terminology is used herein for convenience only and is not to be taken as a limitation on the invention. For example, words such as “upper,” “lower,” “left,” “right,” “horizontal,” “vertical,” “upward,” and “downward” merely describe the configuration shown in the FIGs. Indeed, the components may be oriented in any direction and the terminology, therefore, should be understood as encompassing such variations unless specified otherwise.

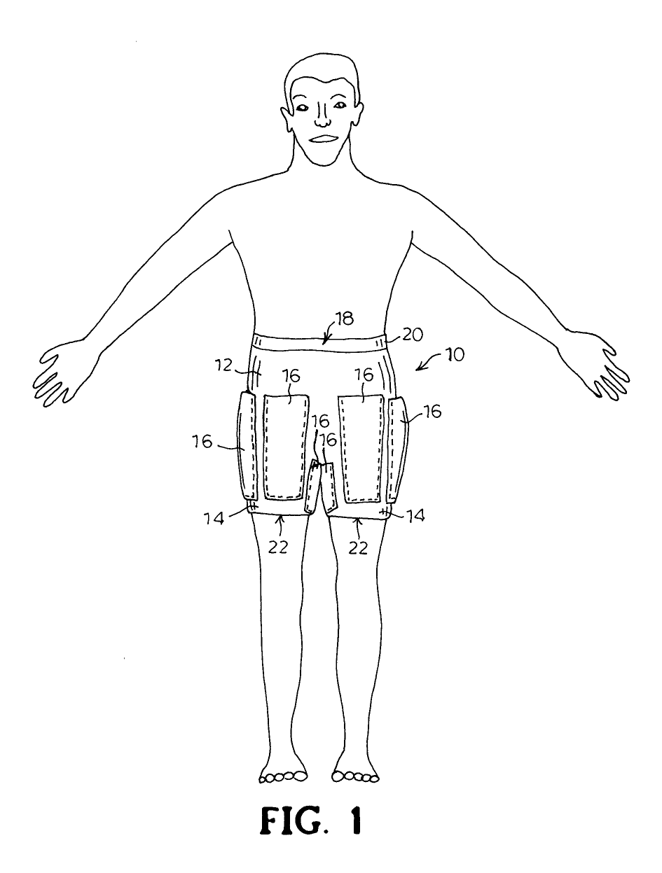

Referring now to the drawings, wherein like reference numerals designate corresponding or similar elements throughout the several views, a therapy garment is shown for providing localized temperature and pressure therapy to a region of a body of a user and is generally designated at 10. Referring to FIGS. 1-4, the therapy garment 10 comprises a main body member 12, a pair of leg sections 14, and a plurality of pockets 16 spaced apart on the body member 12. The main body member 12 is generally tubular for encircling the lower torso in the hip region of the user. The upper portion of the main body member 12 has a torso opening 18 defined by the upper edge of the main body member 12 and adapted to accommodate the torso of the user. As seen in the FIGs., the torso opening 18 may be defined by a waist band 20. The lower portion of the main body member 12 has a pair of openings defined by the lower edge of the main body member. The pair of openings are adapted to accommodate the legs of the user and are appropriately sized and positioned so that the legs of the user may comfortably extend through the main body member 12. The main body member 12 may include a conventional closure (not shown), such as a zipper, hook, pile closure, or the like, whereby the user may conveniently get into or out of the therapy garment.

The pair of leg sections 14 are attached to the main body member 12 at the pair of openings in the lower portion of the body member 12 such that the leg sections 14 depend from the main body member 12. Preferably, the therapy garment 10 is formed from one integral piece of fabric, creating a seamless junction between the main body member 12 and the pair of leg sections 14. Alternatively, the leg sections 14 may be attached to the main body member 12 using any suitable means, such as by sewing. The pair of leg sections 14 are generally tubular and configured to surround the legs of the user. Each of the leg sections 14 has an opening at the distal end defined by the lower edge of the leg section 14. The leg section 14 openings are adapted to pass the feet and legs of the user and are appropriately sized and positioned so that the feet and legs of the user may comfortably extend through the leg sections 14.

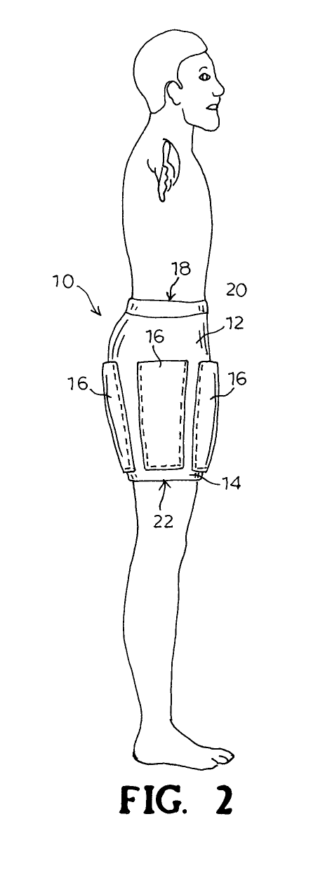

As shown in FIGS. 1 and 2, when the therapy garment 10 is worn by the user, the therapy garment 10 extends downwardly from just below the rib cage of the user. In the embodiment shown, the pair of leg sections 14 extend downwardly from the crotch of the user to above the knees. In an alternative embodiments, the pair of leg sections 14 may extend downwardly from the crotch to immediately below the knees, to the ankles, or to any location between the crotch and the bottom of the feet.

In one embodiment of the therapy garment 10, the pockets 16 are provided in predetermined locations on an outer surface of the therapy garment 10. The pockets 16 may be permanently affixed to the main body member 12 or the pair of leg sections 14 using any suitable means for attachment. For example, the pockets 16 may be integrally formed with the therapy garment 10, whereby the material of the therapy garment 10 is next to the skin of the user and the pockets 16 are formed by a second sheet, which is stitched to the therapy garment 10 to form a seam. Alternatively, the pockets 16 may be attached to the main body member 12 or pair of leg sections 14 using a thermal or heat-sensitive adhesive. It is understood that any other suitable fastening technique may be used for securing the material of the pockets 16 to the material of the therapy garment 10.

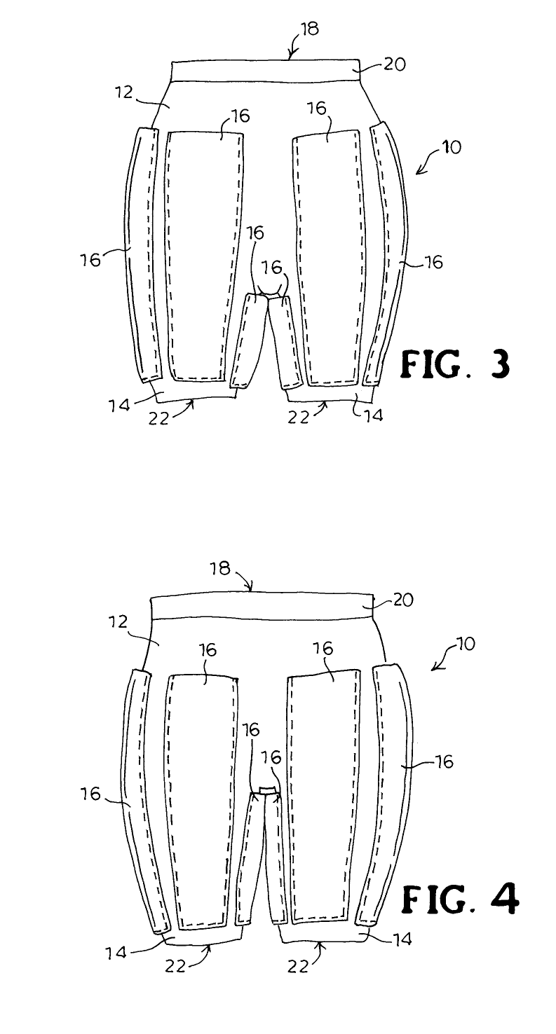

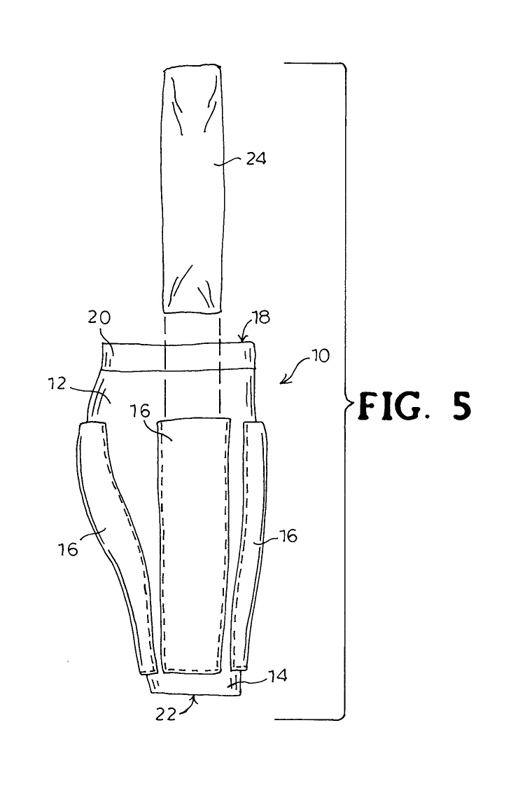

The location of a pocket 16 on the outer surface of the therapy garment 10 is adapted for aligning the pocket 16 on the region of the body of the user that is to receive temperature therapy. The location may be determined based on the anticipated use of the therapy garment 10. In one embodiment, the therapy garment 10 may be used for recovery after exercise. In this embodiment, the plurality of pockets 16 are provided and aligned with major muscle groups in the lower body. Referring to FIGS. 3 and 4, the pockets are aligned with the quadriceps and lower abdominal muscles, the hamstrings and lower back muscles, the inner thighs and the outer thighs muscles. In the embodiment of the therapy garment 10 wherein the leg sections 14 extend to at least the calves, additional pockets 16 may be aligned with the calf muscles (not shown). More specifically, the muscles of the user that may receive temperature or pressure therapy using the therapy garment 10 include the adductor longus, adductor magnus, biceps femoris, extensor digitorum longus, extensor hallucis longus, external oblique, gastrocnemius, gluteus maximum, gluteus medius, gracilis, iliopsoas, pectineus, peroneus brevis, peroneus longus, plantaris, rectus femoris, sartorius, semimembranosus, semitendinosus, soleus, tensor fasciae latae, tibialis anterior, vastus lateralis, and vastus medialis muscles.

The therapy garment 10 may also be intended to be used for recovery from an injury or a surgery and will have one or more pockets 16 aligned with the region in need of recovery. For example, if the therapy garment 10 is intended to assist in recovery from a groin injury, at least one pocket 16 may be aligned with the region of the interior thigh that corresponds with the major groin muscles.

The therapy garment 10 includes a temperature therapy element 24 disposed in a pocket 16. The temperature therapy element 24 may be either heated or cooled, depending on whether the muscle or injury requires therapeutic heating or cooling. The temperature therapy element 24 may come in many shapes and sizes adapted to fit in the pockets 16 and provide temperature therapy through the material of the therapy garment 10. In one embodiment, the temperature therapy element 24 may be of a type that provides either heating or cooling, depending upon the treatment of the temperature therapy element 24 before it is inserted into the pocket 16. For example, commercially available temperature therapy elements may be cooled in a freezer or heated in a microwave to provide the temperature therapy desired. It is understood that the specific nature of the removable temperature therapy element 24 is not a critical aspect of the present invention and many types of temperature therapy elements known in the art may be suitable.

The shape and size of each pocket 16 is adapted for receiving the intended temperature therapy element 24. The pockets 16 are adapted so that the contents of the pocket 16 are thermally connected to the body. This is accomplished by the pocket 16 holding the contents snugly against the body of the user, or even enhanced by forming the material defining the pocket 16 from a thermoconductive material that transmits the temperature of the therapy element to the skin. In one embodiment, the temperature therapy element 24 is removable and may be selectively inserted and then removed from the pocket 16.

In another embodiment, one or more of the pockets 16 of the therapy garment 10 may optionally be closable. For example, after a removable temperature therapy element 24 is inserted into the pocket 16, the open edge of that pocket 16 is closed by suitable fastening means. The closure is adapted to prevent the removable temperature therapy element 24 from exiting the pocket 16 unintentionally. Preferably, the pocket 16 may be closed by a flap (not shown) that is attached to the main body member 12 or a leg section 14. The flap is configured to be positioned over the open edge of the pocket 16. For example, the flap may be removably attached to the pocket 16 by means of fastener strips such as VELCRO so that the pocket 16 is closed. Alternatively, the flap may be attached to the pocket by means of adhesive strips, snaps, buttons, hook and loop type fasteners, a zipper, a magnetic closure mechanism, or any other appropriate closure device known in the art. The pocket 16 may be closed by means other than a flap as well, such as by means of a zipper, and such an embodiment is within the scope of the therapy garment 10 described herein.

The material of the therapy garment 10 is not a critical aspect of the invention. Preferably, the therapy garment 10 is manufactured of a generally breathable, resilient, substantially flexible material. The material may flex with the movement of the body so that the user may be mobile while wearing the therapy garment. The resilient flexibility of the therapy garment 10 may also provide support for the muscles of the abdomen, lower back, and thighs because of the tension of the fabric against the skin. In one embodiment, the therapy garment 10 comprises one or more highly compression regions for applying therapeutic pressure to specific locations on the body of the user. In a preferred embodiment, the compression regions are integral with the material of the therapy garment 10. Preferably, the material of the therapy garment 10 is washable. Suitable materials for the therapy garment may include neoprene, nylon, spandex, lycra, or any other stretchable fabric.

The material may also wick moisture away from the body so that the temperature therapy does not produce moisture next to the skin. The material may be adapted so that the therapy garment 10 conforms to the body and prevents the temperature therapy element 24 from shifting from the predetermined location. In this aspect, the therapy garment 10 may provide a compressive force on the body of the user when worn. As described above, in a preferred embodiment the therapy garment 10 is comprised of a thermoconductive material at least where the pocket 16 is attached to the main body member 12 or pair of legs section 14 so that the temperature therapy element 24 efficiently transfers temperature to the region of the body selected to receive temperature therapy.

The embodiments and examples presented herein are illustrative of the general nature of the subject matter claimed and are not limiting. It will be understood by those skilled in the art how these embodiments can be readily modified and/or adapted for various applications and in various ways without departing from the spirit and scope of the subject matter disclosed. The claims hereof are to be understood to include without limitation all alternative embodiments and equivalents of the subject matter hereof. Phrases, words and terms employed herein are illustrative and are not limiting. Where permissible by law, all references cited herein are incorporated by reference in their entirety. It will be appreciated that any aspects of the different embodiments disclosed herein may be combined in a range of possible alternative embodiments, and alternative combinations of features, all of which varied combinations of features are to be understood to form a part of the subject matter claimed.

I claim:

1. A therapy garment for applying localized temperature therapy to a body of a user, the therapy garment comprising:
a tubular main body member defining a torso opening and a pair of openings for accommodating a pair of limbs of a user, the main body member encircling the torso of a user;
a pair of tubular leg sections attached to the portion of the main body member defining the pair of openings, the pair of leg sections encircling a pair of legs of the user; and
at least one pocket provided on the main body member or the pair of leg sections and adapted for overlying a region on the user to receive temperature therapy.

2. The therapy garment of claim 1, further comprising a temperature therapy element that fits within the at least one pocket.

3. The therapy garment of claim 2, wherein the temperature therapy element is selectively insertable and removable from the at least one pocket.

4. The therapy garment of claim 2, wherein the temperature therapy element may be selectively heated or cooled according to whether the localized temperature therapy entails application of heating or cooling to the region of the body of the user to receive temperature therapy.

5. The therapy garment of claim 2, wherein the temperature therapy element is thermally connected to the body of the user.

6. The therapy garment of claim 1, wherein the main body member, the pair of leg sections, and the at least one pocket are formed from a generally breathable, resilient, substantially flexible material.

7. The therapy garment of claim 1, wherein the pair of leg sections are sized so as to extend to the ankles of the user.

8. The therapy garment of claim 1, wherein the at least one pocket is formed by a panel of resilient fabric secured to the main body member or the pair of leg sections.

9. The therapy garment of claim 8, wherein the at least one pocket is secured to the outer surface of the main body member or the pair of leg sections.

10. The therapy garment of claim 1, wherein the region on the user to receive temperature therapy corresponds to a muscle group of the user selected from the lower back, abdominal region, outer region of thighs, inner region of thighs, or calves.

11. The therapy garment of claim 10, wherein the muscle group comprises at least one of the adductor longus, adductor magnus, biceps femoris, extensor digitorum longus, extensor hallucis longus, external oblique, gastrocnemius, gluteus maximum, gluteus medius, gracilis, iliopsoas, pectineus, peroneus brevis, peroneus longus, plantaris, rectus femoris, sartorius, semimembranosus, semitendinosus, soleus, tensor fasciae latae, tibialis anterior, vastus lateralis, and vastus medialis muscles.

12. The therapy garment of claim 1, wherein the at least one pocket includes a means for selectively closing and opening the at least one pocket.

13. The therapy garment of claim 12, wherein the means for selectively closing and opening the at least one pocket is selected from a zipper, a flap, a button, a hook and loop, a magnetic fastener, and a Velcro attachment.

14. The therapy garment of claim 1, further comprising a compression region for applying pressure therapy to the body of the user.

15. The therapy garment of claim 14, wherein the compression region comprises compression material for form-fitting the body of the user.

16. The therapy garment of claim 15, wherein the compression material comprises at least one of neoprene, nylon, spandex, and lycra.
